(19) United States
(12) Patent Application Publication
(10) Pub. No.: US 2005/0182039 A1
(43) Pub. Date: Aug. 18, 2005

(54) Use of Loteprednol etabonate for the treatment of dry eye

(75) Inventor: Robert J. Meyering, Tampa, FL (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/056,643

(22) Filed: Feb. 11, 2005

Related U.S. Application Data
(60) Provisional application No. 60/544,505, filed on Feb. 13, 2004.

(51) Int. Cl. A61K 31/57; A61K 9/14

(52) U.S. Cl. 514/178; 424/486

(57) ABSTRACT

Disclosed in embodiments herein is a method of treating dry eye in a patient in need thereof, the method comprising topically administering to the patient Loteprednol etabonate in an ophthalmolically acceptable carrier.

### CROSS REFERENCE TO RELATED APPLICATIONS

This application claims the benefit of Provisional Patent Application No. 60/544,505 filed Feb. 14, 2004 and is incorporated herein by reference.

### BACKGROUND AND SUMMARY

Dry eye, also known generically as keratoconjunctivitis sicca (KCS), is a common ophthalmological disorder affecting millions of Americans each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease manifest dry eye complications.

The human ocular surface is normally covered by a tear film that is composed of a superficial thin lipid layer (primarily derived from meibomian gland secretions), a middle bulk aqueous layer (consisting of proteins, electrolytes, and water secreted by lacrimal glands), and the innermost mucus layer derived from mucins secreted by the ocular surface epithelial cells.

A stable tear film ensures comfort and serves as the refractive optical surface of the eye. Furthermore, the tear film serves as a barrier for the ocular surface against microbial infection and inflammation from mechanical trauma. Tear film deficiencies, referred to as dry eye, are a common clinical problem that can result from decreased secretion of tears by the lacrimal gland and/or increased evaporative loss due to a deficient lipid layer or blink abnormalities. Patients with mild dry eye complain of annoying eye irritations. Those with severe dry eye, such as Sjogren's syndrome, may experience constant and disabling eye irritation, and develop ocular surface epithelial disease and sight-threatening sterile or microbial corneal ulceration. The other reason that ocular irritation can occur is when the clearance of tears is delayed. Delayed tear clearance can be found in a number of other ocular surface disorders. Delayed tear clearance results in the accumulation of ocular irritants which can be derived from the environment (pollutants), medications (or preservatives), and cells (inflammatory mediators).

Corticosteroids are potent, non-specific anti-inflammatory drugs that inhibit a variety of chemotactic substances and factors that mediate capillary permeability, contraction of nonvascular smooth muscle, and vasodilatation. In addition, corticosteroids suppress inflammation by inhibiting edema, fibrin deposition, migration of leukocytes and phagocytic activity.

Topical corticosteroids are useful in a variety of ophthalmic conditions and are generally indicated for treatment of steroid-responsive inflammatory conditions of the palpebral and bulbar conjunctiva, cornea and anterior segment of the eye. Although corticosteroids are widely used as a topical agent for ocular inflammation, most possess a safety risk profile that limits their more general utility. A common risk associated with corticosteroid therapy is an elevation of intraocular pressure (IOP). In addition, chronic use of topical corticosteroids may result in the development of cataracts. Loteprednol etabonate is a compound designed as a site-active corticosteroid that will undergo a predictable transformation to an inactive metabolite. The relatively rapid metabolism of Loteprednol etabonate to an inactive metabolite improves the safety profile of this corticosteroid. This characteristic of Lotemax® (Loteprednol etabonate ophthalmic suspension, 0.5%) makes it an excellent candidate for use in inflammatory ocular conditions.

For years it has been recognized that patients with dry eye develop pathologic changes of the ocular surface epithelial cells termed squamous metaplasia. Unreported research suggests that this process is the result of increased proliferation, abnormal differentiation, and inflammation of the ocular surface epithelial cells. In contrast to normal cells, these metaplastic cells do not produce the mucus that normally coats the ocular surface and forms a barrier against infection and mechanical trauma. This renders the ocular surface susceptible to damage from the mild trauma of desiccation blinking, rubbing, and foreign bodies (such as contact lenses).

Lotemax® (Loteprednol etabonate ophthalmic suspension, 0.5%) was approved by the FDA in March, 1998 for use in the treatment of steroid responsive conditions. Researchers have concluded that there is an inflammatory etiology in some, if not most, cases of dry eye. Reports of clinical studies have supported the use of topical corticosteroid solutions in the treatment of patients with keratoconjunctivitis sicca. Stephen Pflugfelder, MD, introduced the diagnostic test of fluorescein tear clearance to further differentiate and select patients with dry eye with an inflammatory component. It is postulated that the reduced flow of tears through the tear film and out of the cul-de-sac into the naso-lacrimal duct results in stasis, which allows inflammatory mediators in the tear film to remain in contact with the mucosa of the conjunctiva and the corneal epithelium. Stasis, thus, may increase the inflammation associated with the KCS. This process may also be responsible for a further reduction of tear production in such patients.

Although dry eye has not traditionally been considered to be an inflammatory disease, the following recent patents or publications are noted:

U.S. Pat. No. 6,153,607 discloses a preservative-free composition containing an effective amount of a corticosteroid in an aqueous carrier to treat a dry eye condition. The composition may be provided as a part of a therapeutic regimen to treat a variety of dry eye conditions and ocular surface disorders manifesting delayed tear clearance previously not readily treatable. The composition may be packaged as containers of single dosage amounts of the corticosteroid-aqueous composition sufficient for pulsed-therapy of acute exacerbations of the irritation symptoms and ocular surface disease of conditions associated with dry eye and delayed tear clearance.

U.S. Patent Application. Publication No. 20030008853 discloses 22,29-epoxy-3,4,6,7,29-pentahydroxy-,(3α,4β,5α,6α,7-β,14β,22S)-stigmastan-15-one as useful for treating dry eye disorders and other disorders requiring the wetting of the eye. This publication further notes that corticosteroids, such as prednisolone, dexamethasone, fluoromethalone, hydrocortisone, loteprednol, triamcinolone, etc., cannot be used for prolonged therapy in dry eye patients without causing side effects. This publication further notes that steroid-related complications including increased intraocular pressure and cataract formation have been observed in dry eye patients treated with corticosteroids after several months of therapy. Therefore, it was surprisingly discovered that a topical ophthalmic formulation containing Loteprednol etabonate, when used for four weeks, was efficacious and well tolerated in the management of patients with KCS with inflammation. Moreover, a differential treatment effect, which was in some cases significant, was seen in subsets of patients who presented a moderate to severe inflammatory component corresponding with more severe KCS.

Therefore, disclosed in embodiments herein is a method of treating dry eye in a patient in need thereof, the method comprising topically administering to the patient Loteprednol etabonate in an ophthalmologically acceptable carrier.

### DETAILED DESCRIPTION

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

Topical steroids for treating ocular inflammations can be based on predictably metabolized drugs. Predictably metabolized drugs, as is known in the art, are designed to provide maximal therapeutic effect and minimal side effects. By one approach, synthesis of a “predictably metabolized drug” can be achieved by structurally modifying a known inactive metabolite of a known active drug to produce an active metabolite that undergoes a predictable one-step transformation in-vivo back to the parent, inactive metabolite (see, U.S. Pat. Nos. 6,610,675, 4,996,335 and 4,710,495 for predictably metabolized steroids). “Predictably metabolized drugs” therefore are biologically active chemical components characterized by predictable in vivo metabolism to non-toxic derivatives after they provide their therapeutic effect. Formulations of steroids suitable for ophthalmic use are known. For example, U.S. Pat. Nos. 4,710,495, 4,996,335, 5,540,930, 5,747,061, 5,916,550, 6,368,616 and 6,610,675, the contents of each of which is incorporated by reference herein, describe predictably metabolized steroids and formulations containing predictably metabolized steroids.

(11β,17α),-17-[(Ethoxycarbonyl)oxy]-11-hydroxy-3-oxoandrosta-1,4-diene-17-carboxylic acid chloromethyl ester (loteprednol etabonate) is a known compound and can be synthesized by methods disclosed in U.S. Pat. No. 4,996,335, the entire contents of which are hereby incorporated by reference in the present specification.

According to the methods of the present invention, a composition comprising (11β,17α),-17-[(Ethoxycarbonyl)oxy]-11-hydroxy-3-oxoandrosta-1,4-diene-17-carboxylic acid chloromethyl ester and a pharmaceutically acceptable carrier for topical ophthalmic administration or implantation into the conjunctival sac or anterior chamber of the eye is administered to a mammal in need thereof. The compositions are formulated in accordance with methods known in the art for the particular route of administration desired.

The compositions administered according to the present invention comprise a pharmaceutically effective amount of (11β,17α),-17-[(Ethoxycarbonyl)oxy]-11-hydroxy-3-oxoandrosta-1,4-diene-17-carboxylic acid chloromethyl ester. As used herein, a “pharmaceutically effective amount” is one which is sufficient to reduce or eliminate signs or symptoms of dry eye or other disorders requiring the wetting of the eye. Generally, for compositions intended to be administered topically to the eye in the form of eye drops or eye ointments, the amount of (11β,17α),-17-[(Ethoxycarbonyl)oxy]-11-hydroxy-3-oxoandrosta-1,4-diene-17-carboxylic acid chloromethyl ester will be about 0.001 to 5.0% (W/W). For preferred topically administrable ophthalmic compositions, the amount of (11β,17α),-17-[(Ethoxycarbonyl)oxy]-11-hydroxy-3-oxoandrosta-1,4-diene-17-carboxylic acid chloromethyl ester will be about 0.001 to 1.0% (W/W).

The compositions administered according to the present invention may also include various other ingredients, including but not limited to surfactants, tonicity agents, buffers, preservatives, co-solvents and viscosity building agents.

Surfactants that can be used are surface-active agents that are acceptable for ophthalmic or otolaryngological uses. Useful surface active agents include but are not limited to polysorbate 80, tyloxapol, TWEEN 80 (ICI America Inc., Wilmington, Del.), PLURONIC F-68 (from BASF, Ludwigshafen, Germany) and the poloxamer surfactants can also be used. These surfactants are nonionic alkaline oxide condensates of an organic compound which contains hydroxyl groups. The concentration in which the surface active agent may be used is only limited by neutralization of the bactericidal effects on the accompanying preservatives (if present), or by concentrations which may cause irritation.

Various tonicity agents may be employed to adjust the tonicity of the composition. For example, sodium chloride, potassium chloride, magnesium chloride, calcium chloride, nonionic diols, preferably glycerol, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% W/W. Unit dose compositions of the present invention will be sterile, but typically unpreserved. Such compositions, therefore, generally will not contain preservatives.

Co-solvents and viscosity building agents may be added to the compositions to improve the characteristics of the compositions. Such materials can include nonionic water-soluble polymer. Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P. Other compounds may also be added to the ophthalmic compositions of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Compositions formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous compositions which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of (11β,17α),-17-[(Ethoxycarbonyl)oxy]-11-hydroxy-3-oxoandrosta-1,4-diene-17-carboxylic acid chloromethyl ester. Examples of artificial tears compositions useful as artificial tears carriers include, but are not limited to, commercial products, such as Moisture Eyes™ Lubricant Eye Drops/Artificial Tears, Moisture Eyes™ Liquid Gel lubricant eye drops, Moisture Eyes™ Preservative Free Lubricant Eye Drops/Artificial Tears and Moisture Eyes™ Liquid Gel Preservative Free Lubricant Eye Drops/Artificial Tears (Bausch & Lomb Incorporated, Rochester, N.Y.). Examples of phospholipid carrier formulations include those disclosed in U.S. Pat. No. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Glonek et al.), the contents of each of which are incorporated by reference herein.

Compositions formulated for the treatment of dry eye-type diseases and disorders may be prepared as ophthalmic ointments. Ointments are well known ophthalmic compositions and are essentially an oil-based delivery vehicle. Typical ointments use petroleum and/or lanolin base to which is added the active ingredient, usually as 0.1 to 2%, and excipients. Common bases include mineral oil, petrolatum and combinations thereof, but oil bases are not limited thereto.

The preferred compositions of the present invention are intended for administration to a human patient suffering from dry eye or symptoms of dry eye. Preferably, such compositions will be administered topically. In general, the doses used for the above described purposes will vary, but will be in an effective amount to eliminate or improve dry eye conditions. Generally, 1-2 drops of such compositions will be administered from once to many times per day. The composition is intended to be provided as a package for the treatment of dry eye, the package would include the pharmaceutical formulation comprising Loteprednol etabonate contained in a pharmaceutically acceptable container; a written package insert containing instructions for using the formulation for the treatment of dry eye; and outer packaging identifying the pharmaceutical formulation contained therein. In certain embodiments wherein the composition is preservative free the package would contain a pharmaceutically acceptable container suitable for single use by a user of the packaged composition. In such embodiments it is envisioned that the outer packaging would contain at least one pharmaceutically acceptable container containing the Loteprednol etabonate composition. Preferably the outer packing would contain a multiplicity of single use containers, for example, enough single use containers to provide for a one-month supply of the composition.

The invention will now be further described by way of several examples that are intended but not limit the scope of the invention as defined by the claims herein.

Representative eye drop formulations are provided in Examples 1-4 below.

### EXAMPLE 1

| | Lotemax ® (loteprednol etabonate | |
|---|---|---|
| Ingredients (per mL) | ophthalmic suspension, 0.5%) | |
| Loteprednol etabonate, NDS, | 5.00 | mg |
| micronized, sterile | | |
| Povidone, USP(C-30) | 6.00 | mg |
| Benzalkonium chloride, 50% | 0.20 | mg |
| solution NF | | |
| Edetate disodium dihydrate, | 0.10 | mg |
| USP | | |
| Glycerin, USP, 96% | 25.00* | mg |
| Tyloxapol, USP | 3.00 | mg |
| Purified water, USP | QS to 1 | mL |

| | |
|---|---|
| Hydrochloric acid, 37%, NF | Adjust pH |
| (diluted to 0.1 N) | |
| Sodium hydroxide, NF | Adjust pH |
| (diluted to 0.1 N) | |

*25.0 mg/mL of glycerin, 96% is equivalent to 24 mg/mL (2.4 W/W) of glycerin, 100%

### EXAMPLE 2

| Ingredient | Amount |
|---|---|
| Phase I | |
| Carbopol 934P NF | 0.25 gm |
| (Acrylic acid-based polymer) | |
| Purified Water | 99.75 gm |
| Phase II | |
| Propylene Glycol | 5.0 gm |
| EDTA | 0.1 mg |
| Loteprednol Etabonate | 50.0 gm |

Mix five parts of phase II with twenty parts of phase I for more than 15 minute and adjust pH to 6.2-6.4 using 1 N NaOH.

### EXAMPLE 3

| Ingredient | Amount |
|---|---|
| Phase I | |
| Carbopol 934P NF | 0.25 gm |
| (Acrylic acid-based polymer) | |
| Purified Water | 99.75 gm |
| Phase II | |
| Propylene Glycol | 3.0 gm |
| Triacetin | 7.0 gm |
| Loteprednol Etabonate | 50.0 gm |
| EDTA | 0.1 gm |

Mix five parts of phase II with twenty parts of phase I for more than 15 minutes and adjust pH to 6.2-6.4 using 1 N NaOH.

### EXAMPLE 4

| Ingredient | Amount |
|---|---|
| Phase I | |
| Carbopol 934P NF | 0.25 gm |
| (Acrylic acid-based polymer) | |
| Purified Water | 99.75 gm |
| Phase II | |
| Propylene Glycol | 7.0 gm |
| Glycerin | 3.0 mg |
| Loteprednol Etabonate | 50.0 gm |
| EDTA | 0.1 mg |
| BAK | 01-0.2 mg |

Mix five parts of phase II with twenty parts of phase I for more than 15 minutes and adjust pH to 6.2-6.4 using 1 N NaOH.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

1. A method for the treatment of dry eye compromising: administering to a mammal a composition comprising a pharmaceutically acceptable carrier and a dry eye treatment effective amount of (11β,17α),-17-[(Ethoxycarbonyl)oxy]-11-hydroxy-3-oxoandrosta-1,4-diene-17-carboxylic acid chloromethyl ester.

2. The method of claim 1 wherein the pharmaceutically effective amount of (11β,17α),-17-[(Ethoxycarbonyl)oxy]-11-hydroxy-3-oxoandrosta-1,4-diene-17-carboxylic acid chloromethyl ester is between 0.001-5.0% (W/W).

3. The method of claim 2 wherein the pharmaceutically effective amount of (11β,17α),-17-[(Ethoxycarbonyl)oxy]-11-hydroxy-3-oxoandrosta-1,4-diene-17-carboxylic acid chloromethyl ester is between 0.001-1.0% (W/W).

4. The method of claim 1 wherein the composition is topically administered to the eye.

5. The method of claim 1 wherein the dry eye is diagnosed by symptoms of dry eye associated with refractive surgery.

6. The method of claim 1 wherein the composition is an ophthalmic suspension.

7. The method of claim 6 wherein the ophthalmic suspension comprises 0.5 wt percent of Loteprednol etabonate in a pharmaceutical carrier comprising povidone, benzalkonium, chloride, disodium EDTA, glycerin, tyloxapol and water.

8. The method of claim 1 wherein the composition is a gel.

9. The method of claim 8 wherein the gel compromises acrylic acid-based polymer, water, propylene glycol, EDTA and Loteprednol etabonate.

10. The method of claim 9 wherein the gel further compromises triacetin.

11. The method of claim 8 wherein the composition compromises acrylic acid-based polymer, water, propylene glycol, glycerin, EDTA, benzalkonium chloride and Loteprednol etabonate.

12. An ophthalmic gel formulation suitable for the treatment of dry eye comprising Loteprednol etabonate and an acrylic-based polymer.

13. The formulation of claim 12 further comprising at least one member selected from the group consisting of water, propylene glycol, EDTA, triacetin and benzalkonium chloride.

14. A package for the treatment of dry eye, the package comprising:
a pharmaceutical composition comprising Loteprednol etabonate contained in a pharmaceutically acceptable container;
a written package insert containing instructions for using the composition for the treatment of dry eye; and
outer packaging identifying the pharmaceutical composition contained therein.

15. The package of claim 14 wherein the pharmaceutically acceptable container is suitable for single use by a user of the composition contained in the package.

16. The package of claim 15 wherein the outer packaging contains at least one pharmaceutically acceptable container containing the Loteprednol etabonate pharmaceutical composition.
